(19) United States
(12) Patent Application Publication
(10) Pub. No.: US 2020/0289062 A1
(43) Pub. Date: Sep. 17, 2020

(54) METHOD OF MANUFACTURING HEAD FIXATION DEVICE COMPONENTS

(71) Applicant: pro med instruments GmbH, Freiburg Im Breisgau (DE)

(72) Inventors: Matthias E. Schuele, Freiburg (DE); Marco S. Willesch, Eichstetten am Kaiserstuhl (DE)

(21) Appl. No.: 16/816,947

(22) Filed: Mar. 12, 2020

Related U.S. Application Data
(60) Provisional application No. 62/818,960, filed on Mar. 15, 2019.

(51) Int. Cl. A61B 5/00; B29C 45/14; B29C 70/16

(52) U.S. Cl. CPC A61B 5/70; B29L 2031/753; B29C 70/16; B29C 45/14631

(57) ABSTRACT

A method or process for making one or more components of a head fixation device includes preforming, molding, and finishing subprocesses. The preforming subprocess includes placing fibers onto a substrate to orient the fibers parallel to a flux of force that the component is expected to experience in use. The fiber layers are combined and can be draped over an optional core before being molded to combine the assembly with a molding material. The resultant molded preform undergoes a finishing subprocess that can include smoothing the component to remove any burrs or sharp edges from the molding subprocess.

### PRIORITY

This application claims priority to U.S. Provisional Patent Application Ser. No. 62/818,960, filed Mar. 15, 2019, entitled “Method of Manufacturing Head Fixation Device Components,” the disclosure of which is incorporated by reference herein.

### BACKGROUND

The devices and methods disclosed pertain to patient stabilization, and in particular head and neck stabilization using stabilization devices known as head stabilization devices, which are also referred to as head fixation devices (hereinafter referred to as “HFDs” or “HFD” in singular). HFDs are sometimes used during a variety of surgical and other medical procedures, for example during head or neck surgery or testing, where it would be desirable to securely hold a patient's head in a certain position. Also, various methods have been used to manufacture HFDs and/or components of HFDs. While a variety of stabilization devices and methods of making stabilization devices have been made and used, it is believed that no one prior to the inventor(s) has made or used an invention as described herein.

### BRIEF DESCRIPTION OF THE DRAWINGS

While the specification concludes with claims which particularly point out and distinctly claim the invention, it is believed the present invention will be better understood from the following description of certain examples taken in conjunction with the accompanying drawings, in which like reference numerals identify the same elements.

FIG. 1 depicts a schematic view of an exemplary process for making fiber reinforced composite parts usable with HFDs described herein.

FIG. 2 depicts a schematic view of an exemplary preforming step of the process of FIG. 1.

FIG. 3 depicts a perspective view of an exemplary layer of fiber placed on a substrate according to the preforming step of FIG. 2.

FIG. 4 depicts a partial cross-sectional side view of an exemplary preform having a plurality of layers of fiber placed on a substrate, as shown in FIG. 3, shown with the plurality of layers draped over an exemplary core and with an insert.

FIG. 5 depicts a schematic view of an exemplary molding step of the process of FIG. 1.

FIG. 6 depicts a partial cross-sectional side view of an exemplary fiber reinforced molded composite after the molding step according to FIG. 5.

FIG. 7 depicts a schematic view of an exemplary finishing step of the process of FIG. 1.

FIG. 8 depicts an exemplary HFD made using the process of FIG. 1.

FIG. 9 depicts a partial cross-sectional view of the HFD of FIG. 8.

FIG. 10 depicts a schematic view of an enlarged internal portion of the HFD of FIG. 8, showing the orientation of the fibers relative to the force applied to the HFD during use.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

### I. Exemplary Process for Making Fiber-Reinforced Components

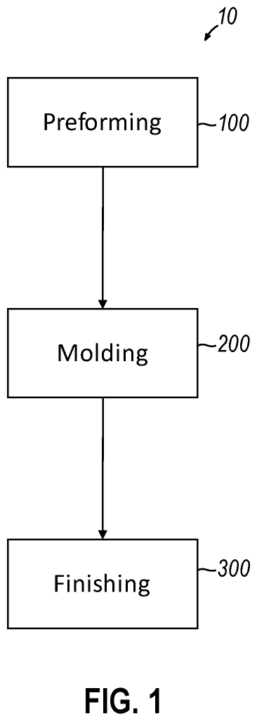

FIG. 1 illustrates a schematic overview of an exemplary process (10) for making components of an HFD. Process (10) allows for the production of fiber-reinforced components that provide high stability and stiffness in a lightweight design. With process (10), fibers can be arranged such that they provide efficient and effective strength and support to withstand forces that the component will be subjected to in use. In other words, by purposeful and strategic arrangement or placement of the fibers used in process (10), a stronger and/or lighter component can be produced. In some instances, this allows for less overall size and/or mass to exist in the component. In the case of medical devices such as HFDs, this can provide for easier imaging techniques and/or better imaging outputs with fewer artifacts. Referring to FIG. 1, process (10) begins with preforming (100), followed by molding (200), and then finishing (300). Each of these subprocesses will be described in greater detail below.

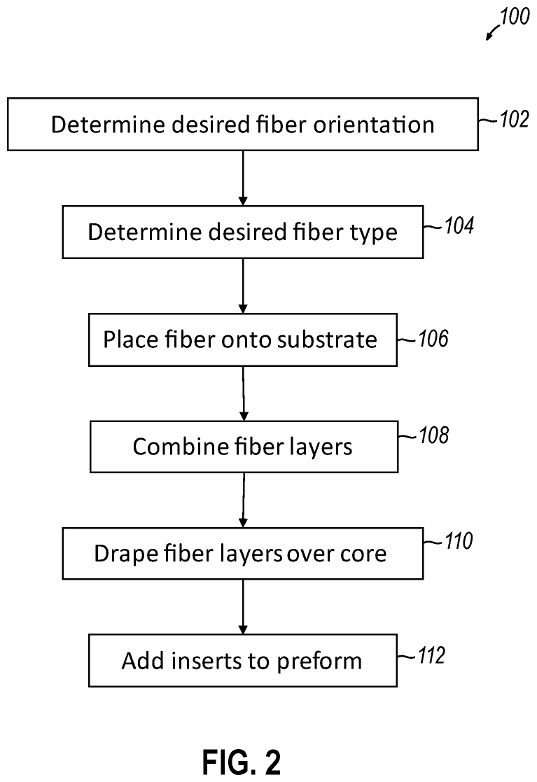

FIG. 2 illustrates a schematic overview of an exemplary preforming (100) step or subprocess as shown in FIG. 1. It should be noted that all the steps shown in FIG. 2 for preforming (100) are not required in all examples. It should further be noted that preforming (100) can include additional steps that may not be shown in FIG. 2, or that may be sub-steps or acts that are included in the steps that are shown in FIG. 2.

Preforming (100) comprises determining a desired fiber orientation (102). When determining the desired fiber orientation (102), the end use of the component being made is considered to understand how the component is subjected to various forces in use. Based on this assessment and understanding, the orientation or arrangement of the fibers can be determined or planned. By way of example only, a component may provide support for an object, or be subject to forces from the object. Thus the object supplies a force or load on the component and this force or load has both a direction and magnitude. The force or load in this respect can also be described as a flux of force, with flux describing the magnitude and direction of the force imparted on the component, or this can be described and/or understood as a force vector where the force vector describes the magnitude and direction of the force.

Factors including, but not necessarily limited to, (1) the geometry of the object, (2) the geometry of the component, (3) the spatial relationship between the object and the component, and (4) how the component and object interact with each other will influence the magnitude and direction of force that is ultimately applied by the object on the component. In some cases, other factors can also influence fiber orientation decisions, such as the thermal expansion and/or stiffness of the materials used in making the component. In view of the teachings herein, various other ways to determine the how forces will impact or influence a given component in use will be apparent to those of ordinary skill in the art.

By way of example, with an understanding and appreciation for the flux of force an object applies on a component in use, the orientation of the fiber in that component can be planned such that the component will provide maximum stiffness and/or strength in a direction parallel to the flux of force that the object applies on the component. More specifically, to achieve these stiffness and/or strength properties in the component, the fibers during preforming (100) are oriented in a direction parallel to the direction of force being applied to the component in use by the object. By orienting the fibers in this manner, a component can be designed and fabricated with efficient strength and/or stiffness properties, meaning that the component provides more strength and/or stiffness with a lower mass of material to provide the necessary or desired strength and/or stiffness in use. This can also mean that the component is designed for the use without the need for costly overengineering the component, which can also have undesirable affects like heavier and/or larger components that make imaging more difficult, etc.

Preforming (100) further comprises determining the desired fiber type (104). By way of example only, and not limitation, some exemplary types of fiber that can be used comprise carbon fiber, glass fiber, aramid fiber, among others. When fabricating a given component one type of fiber can be used for that component, or in other instances more than one type of fiber may be used for that component. By way of example only, fiber type may be selected or determined based at least in part upon the magnitude of the forces expected on a given component in use. For example, in the context of an HFD, one component of the HFD may be subject to greater forces in use than another component of the HFD. Accordingly, in some examples, the component subject to greater forces may use fibers for reinforcement that have stronger and/or stiffer properties to provide for greater strength or stiffness in the component. It should also be understood in this regard that fiber type may refer to the kind of fiber as well as to the size of fiber or length of fiber. In view of the teachings herein, other various fiber types beyond those specifically mentioned that can be used with HFD components made using process (10) will be apparent to those of ordinary skill in the art.

Now that determining the desired fiber orientation (102) and determining the desired fiber type (104) have been explained, other acts in preforming (100), including how the fibers are handled to provide for the desired orientation, will be described. Referring to FIG. 2, preforming (100) further comprises placing fiber onto a substrate (106), such as tissue or other suitable substrate, to form a fiber layer. Placing fiber onto substrate (106) should be understood to include putting the fiber into a particular position, which may be done by simply resting the fiber in a desired position or adhering the fiber in a desired position via mechanical or chemical techniques. For instance, in some versions fiber is stitched onto substrate (106). Still in other versions, fiber may be adhered to substrate (106) using an adhesive. And still in other versions, fiber may simply be set onto substrate (106) without using a mechanical or chemical assistance. In some other versions, substrate may be omitted and fibers are instead placed onto a surface to form a fiber layer such that the surface is not part of the formed fiber layer.

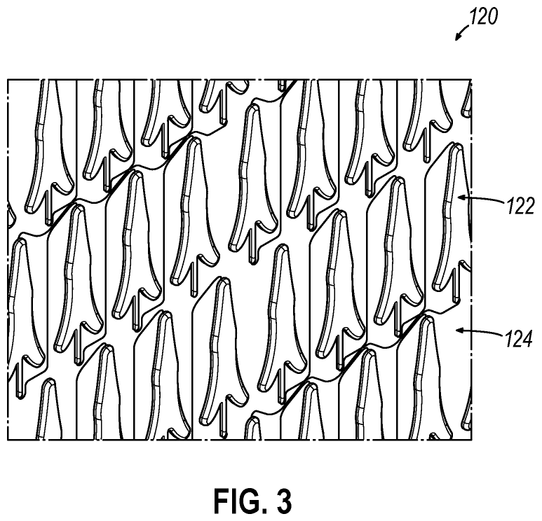

FIG. 3 depicts an exemplary fiber layer (120)—which in the present example comprises a stitched layer—showing fiber (122) stitched on a substrate (124), which in this example is tissue. As shown, fiber layer (120) can include multiple areas where fiber (122) is stitched to substrate (124) in a desired stitch pattern or shape. The stitching in the multiple areas may be identical in the pattern or shape, or the stitching may be different, representing either different stitched layers of the same component or different stitched layers of different components. These multiple areas of fiber layer (120) can be cut out so that each cut-out includes one area where fiber (122) is stitched to substrate (124). Also, in some versions, substrate may be pre-cut or sized so that only one stitched area is included on each fiber layer (120) at the outset.

By controlling the fiber placement in terms of patterns, shapes, and layers, a given finished component can be configured with various fiber types and/or with various fiber orientations. Therefore, while a given component may have a uniform fiber type and/or fiber orientation throughout, this is not required and in other instances a given component can have non-uniform fiber-types and/or fiber orientations throughout.

As mentioned above, where different types of fibers are used within the same component, this may be achieved by using different fiber types for different fiber layers (120). In some instances, fiber being placed can be done such that different fiber types can be used within the same fiber layer (120). This can happen by splicing fiber types together or placing different fibers sequentially within the same fiber layer (120). In view of the teaching herein, various ways to incorporate multiple fiber types into fiber layers (120) and ultimately formed components will be apparent to those of ordinary skill in the art.

As alluded to above, each fiber layer (120) represents a part, section, or slice of the final formed component. Accordingly, with an adequate number of fiber layers (120) for a given component fabricated, preforming (100) further comprises combining fiber layers (108). For instance, a given component may require multiple fiber layers (120) stacked together—and later molded as will be described—to form the desired component. Each fiber layer (120) may be placed and combined considering the orientation of the fiber (122) so that the ultimate formed component is made with the desired fiber orientation and thus strength and/or stiffness properties as discussed above.

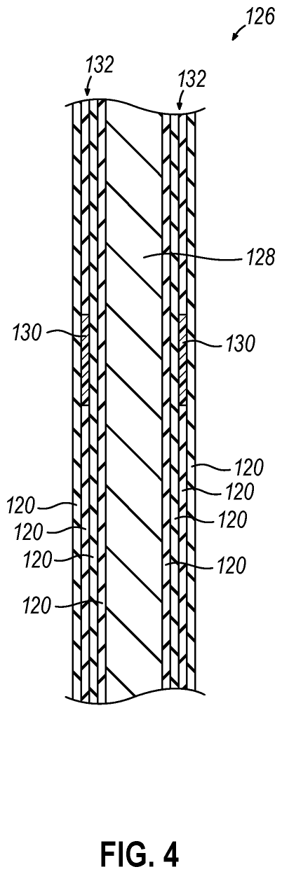

With some components, but not required with all components, preforming (100) comprises draping fiber layers over a core (110) as depicted in FIG. 2. For instance, FIG. 4 depicts a partial cross-sectional side view of an exemplary preform (126) having a plurality of fiber layers (120) shown draped over an exemplary core (128). In this configuration, core (128) carries the fibers (122), which are placed or located in the border or outer regions (132) of the preform (126). In some cases, use of core (128) can be helpful with large-volume components to avoid an excessive use of fiber and/or mass. Of course it should be understood that core (128) can be used with components of any volume.

Core (128) can take a variety of forms. In some examples, core (128) comprises a foam core. Some such foam cores provide for occupying large volumes with little added weight. Other foam cores may be denser in nature however. In some examples, core (128) comprises a honeycomb core. A honeycomb core can provide for a rigid core structure without adding excessive mass to the component. In some examples, core (128) comprises a wool core, while in other examples core (128) comprises a solid core. In view of the teachings herein, various types of materials suitable for use as core (128) will be apparent to those of ordinary skill in the art.

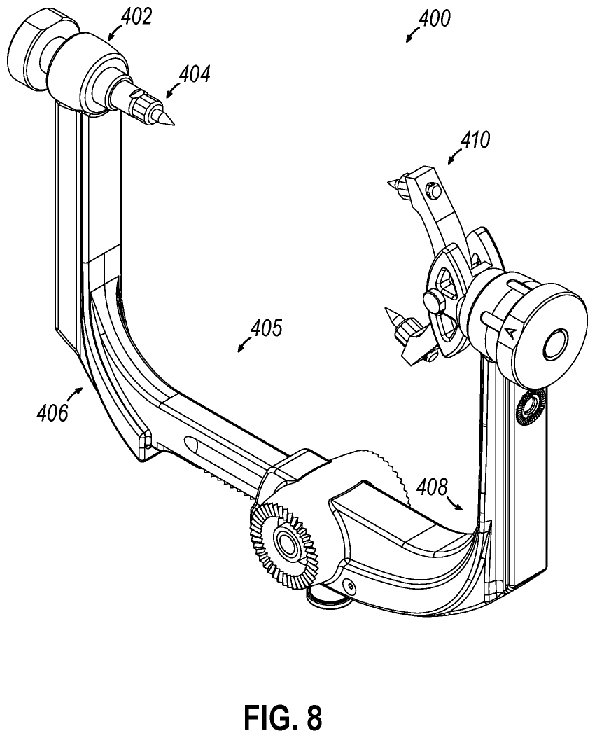

Referring again to preforming (100) and FIG. 2, another optional action with preforming (100) is adding inserts to a preform (112). As shown in FIG. 4, preform (126) can comprise one or more inserts, such as insert (130). Inserts (130) can take a variety of forms. In some examples, insert (130) comprises threads, serrations, or bushings. In this manner the formed component can be strengthened in and/or around these areas with insert (130). For instance, in some example components, insert (130) is included in interface areas where components may be selectively connectable with one another. By way of example and not limitation, an interface area can be one such as interface area (402) as shown in FIG. 8 where a portion of HFD (400) connects with a stabilizing assembly (404). In other examples, insert (130) is included in areas subject to greater forces in use. When placing inserts (130) during preforming (100), inserts (130) can be placed and fiber layers (120) can be placed over and around inserts (130) followed by molding as will be described more below. In view of the teachings herein, other insert types and ways to locate inserts will be apparent to those of ordinary skill in the art.

In some examples preforming (100) can include removal of substrate (124) or excess substrate (124) from fiber layer (120) prior to preform (126) being molded in molding subprocess (200). However, in other examples, such removal of substrate (124) during preforming (100) prior to molding (200) is not required. Where substrate (124) is removed, this removal may be done by cutting, dissolving, burning, or any other suitable way that keeps fiber (122) intact.

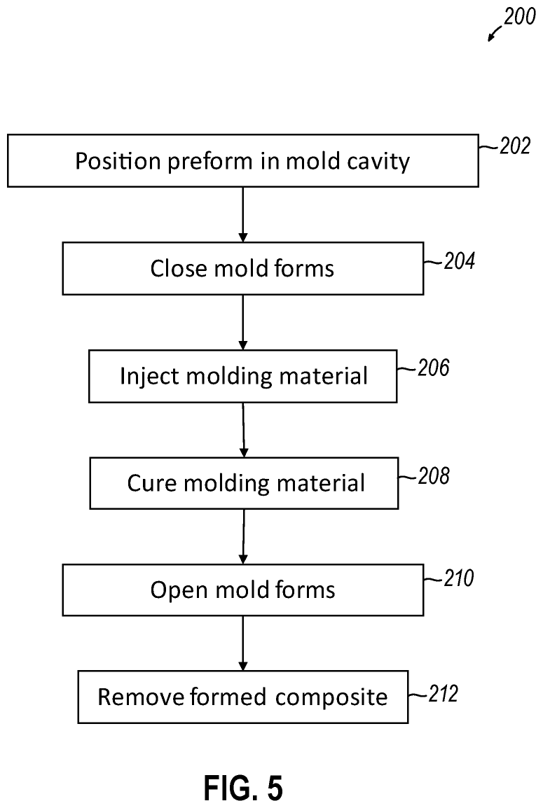

Referring now to FIG. 5, an exemplary molding (200) subprocess is shown, and comprises positioning the preform in the mold cavity (202). With the preform, such as preform (126), within the mold cavity, molding (200) further comprises closing the mold forms (204), and then injecting a molding material within the mold (206). The molding material is liquid resin in some versions, thermoplastic in other versions, and can be other polymeric or other materials in other versions. In one example, the molding material may be a plastic material that melts when heated sufficiently to flow and surround preform (126). Molding (200) further comprises curing the molding material (208), which entails the molding material curing under thermal energy, pressure, and time. Once curing is completed, molding (200) comprises opening the mold forms (210) and removing the formed composite (212) from the mold cavity.

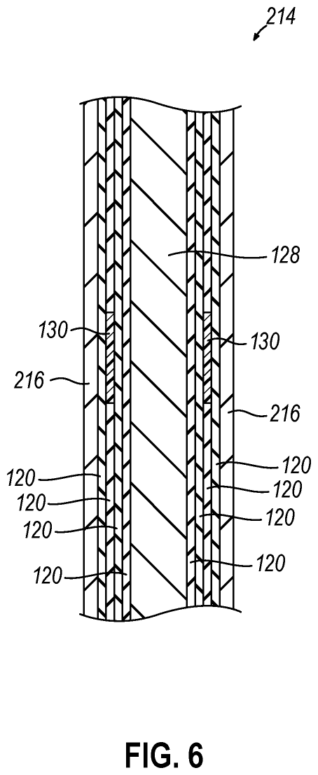

FIG. 6 depicts a partial cross-sectional side view of an exemplary fiber-reinforced molded composite (214) after molding (200) as illustrated in FIG. 5. Composite (214) comprises core (128) as shown in FIG. 4, which is surrounded by fiber layers (120). In the present example shown in FIG. 6, composite (214) also comprises insert (130) as described above. Surrounding fiber layers (120) and insert (130) on the outer surface of composite (214) is plastic layer (216), which represents the cured molding material from molding (200). In some examples, molding (200) can be repeated in a sequential fashion such that more than one plastic layer or molding material layer (216) can surround fiber layers (120). In view of the teachings herein, other ways to modify and/or adapt molding (200) and the resultant composite (214) will be apparent to those of ordinary skill in the art.

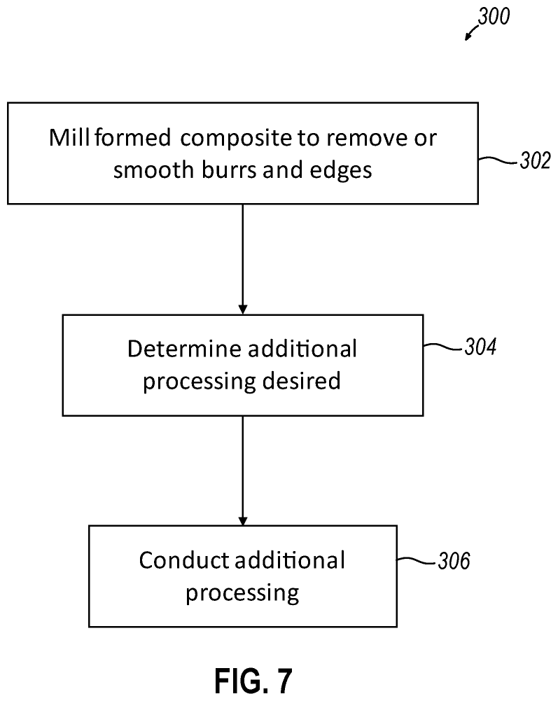

Referring now to FIG. 7, an exemplary finishing (300) subprocess is shown, and comprises milling the formed composite to remove or smooth burrs and edges (302). After molding (200), it can be common for the molded composite (214) to have burrs and edges that result from the parting lines where the molding forms meet. Milling the formed composite (214) removes these burrs and edges. However, other processes like sanding and others that will be apparent to those of ordinary skill in the art in view of the teachings herein can be done instead of or in addition to milling to smooth and/or shape composite (214). Finishing (300) further comprises determining additional processing that may be desired (304) and conducting such additional processing (306). By way of example only, and not limitation, such additional processing can include cutting composites to desired lengths, shaping composites, attaching other structures to composites including other composites by fastening or bonding using adhesives, drilling bores within composites, coating, etc.

FIG. 8 depicts an exemplary HFD (400) in the form of a skull clamp that is made using exemplary process (10). HFD (400) comprises a frame (405) having a first arm or member (406), a second arm or member (408), and a pair of stabilizing assemblies (404, 410). Stabilizing assembly (404) comprises a single pin, and stabilizing assembly (404) is connectable with first arm (406). Stabilizing assembly (410) comprises a dual pin with rocker arm, and stabilizing assembly (410) is connectable with second arm (408). First arm (406) and second arm (408) are adjustably connectable with one another such that the spacing between stabilizing assemblies (404, 410) can be adjusted so HFD (400) can accommodate differing patient head sizes.

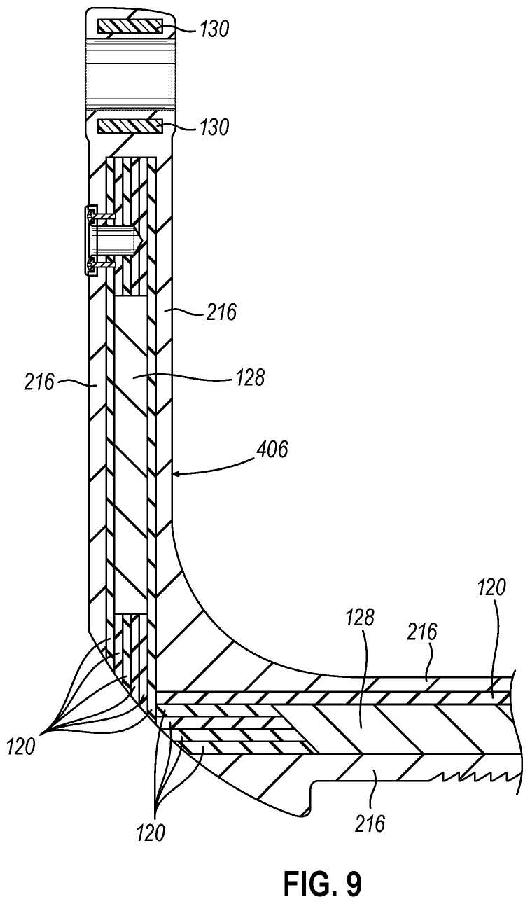

In the present example, one or more components of frame (405), e.g., first arm (406) and/or second arm (408), comprise a fiber-reinforced composite comprising layers of fiber combined with a molding material. Furthermore, such one or more components are configured with the fiber of the fiber-reinforced composite being oriented parallel to a flux of force experienced by the one or more components in use. For example, FIG. 9 depicts a partial cross-sectional view of first arm (406) of HFD (400), showing molding material layer (216) surrounding multiple fiber layers (120) and cores (128) and insert (130). It should be noted that FIG. 9 is exemplary only and that the arrangement of the composite parts is not limited to what is illustrated.

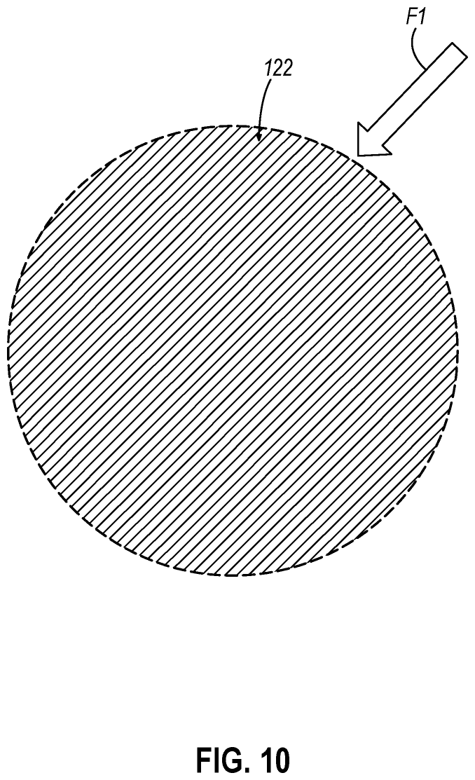

FIG. 10 depicts a schematic view of an enlarged internal portion of HFD (400), showing the orientation of fibers (122) within one of fiber layers (120) relative to the flux of force applied to this portion of HFD (400) during use. As shown, fibers (122) are oriented parallel to the flux of force as indicated by arrow (F1). It should be noted that the flux of force may not be applied in the same manner to all portions of a given component based on factors such as component size and geometry among other things. For instance, at one location within a component the direction of the force applied to the component may differ from the direction of the force applied at another location within the same component. Fiber layers (120) and corresponding fibers (122) within fiber layers (120) are placed within the component such that fibers (122) are generally parallel to the flux of force at the location of the component where fiber layers (120) are placed. In view of the teaching herein, various other ways to orient fibers (122) within fiber layers (120) within a component of an HFD to provide for desired strength and stiffness properties will be apparent to those of ordinary skill in the art.

### II. Exemplary Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

A method of manufacturing a fiber-reinforced component of a head fixation device used in stabilizing a head of a patient during a medical procedure comprises (a) preforming and (b) molding. Preforming comprises (i) placing fiber onto a substrate to form a fiber layer, and (ii) combining multiple fiber layers to form a preform. Molding comprises combining the preform with a molding material to form a molded preform.

### Example 2

The method of manufacturing of Example 1, wherein the act of preforming further comprises determining a desired orientation for the fiber when placing the fiber onto the substrate.

### Example 3

The method of manufacturing of any one or more of Examples 1 through 2, wherein the desired orientation for the fiber is determined by accounting for the stress that is expected to be experienced by the component in use.

### Example 4

The method of manufacturing of any one or more of Examples 1 through 3, wherein the desired orientation for the fiber aligns the fiber parallel to the flux of force that is expected to be experienced by the fiber-reinforced component in use.

### Example 5

The method of manufacturing of any one or more of Examples 1 through 4, wherein the act of preforming further comprises determining a fiber type to be used when placing the fiber onto the substrate to form the fiber layer.

### Example 6

The method of manufacturing of any one or more of Examples 1 through 5, wherein the act of preforming further comprises combining a first fiber layer having a first fiber type with a second fiber layer having a second fiber type, wherein the first and second fiber types are different.

### Example 7

The method of manufacturing of any one or more of Examples 1 through 6, wherein the act of preforming further comprises placing fiber of multiple fiber types onto the substrate to form the fiber layer.

### Example 8

The method of manufacturing of any one or more of Examples 1 through 7, wherein the act of preforming further comprises draping the fiber layers over a core.

### Example 9

The method of manufacturing of Example 8, wherein the core comprises a select one of a foam core, a honeycomb core, a wool core, and a solid core.

### Example 10

The method of manufacturing of any one or more of Examples 8 through 9, wherein the core directs the fibers of the fiber layers so the fibers are placed in a border area of the resulting fiber-reinforced component.

### Example 11

The method of manufacturing of any one or more of Examples 1 through 10 wherein the act of preforming further comprises adding an insert to the preform.

### Example 12

The method of manufacturing of Example 11, wherein the insert is configured to strengthen the resulting fiber-reinforced component.

### Example 13

The method of manufacturing of any one or more of Examples 11 through 12, wherein the insert comprises a select one of threads, serrations, and bushings.

### Example 14

The method of manufacturing of any one or more of Examples 11 through 13, wherein the insert is added at an interface area where multiple fiber-reinforced components are connectable.

### Example 15

The method of manufacturing of any one or more of Examples 1 through 14, wherein the substrate is removed before molding.

### Example 16

The method of manufacturing of any one or more of Examples 1 through 15, wherein the act of molding further comprises placing the preform in a mold cavity and injecting the mold cavity with the molding material.

### Example 17

The method of manufacturing of any one or more of Examples 1 through 16, wherein the act of molding further comprises curing the injected molding material under thermal energy, pressure, and time.

### Example 18

The method of manufacturing of any one or more of Examples 1 through 17, wherein the act of finishing further comprises milling the fiber-reinforced component.

### Example 19

The method of manufacturing of any one or more of Examples 1 through 18, wherein the act of finishing further comprises connecting a first fiber-reinforced component with a second fiber-reinforced component to form an assembly of fiber-reinforced components.

### Example 20

The method of manufacturing of any one or more of Examples 1 through 19, further comprising finishing, wherein the act of finishing comprises smoothing the molded preform to form the fiber-reinforced component.

### Example 21

A head fixation device for use to stabilize a head of a patient in a medical procedure comprises a frame, wherein at least a portion of the frame comprises a fiber-reinforced composite comprising fiber layers combined with a molding material, wherein the fiber of the fiber layers is configured to be oriented parallel to a flux of force experienced by the fiber-reinforced component in use.

### Example 22

The head fixation device of Example 21, wherein the frame comprises (a) a first arm; (b) a second arm selectively and adjustably connectable with the first arm; and (c) a pair of stabilizing assemblies, wherein one of the pair of stabilizing assemblies is selectively and adjustably connectable with the first arm, and wherein the other of the pair of stabilizing assemblies is selectively and adjustably connectable with the second arm.

### Example 23

The head fixation device of any one or more of Example 21 through Example 22, wherein the fiber layers surround a core.

### III. Miscellaneous

It should be understood that any one or more of the teachings, expressions, embodiments, examples, etc. described herein may be combined with any one or more of the other teachings, expressions, embodiments, examples, etc. that are described herein. The following-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We claim:

1. A method of manufacturing a fiber-reinforced component of a head fixation device used in stabilizing a head of a patient during a medical procedure, the method comprising:
(a) preforming, wherein the act of preforming comprises:
(i) placing fiber onto a substrate to form a fiber layer, and
(ii) combining multiple fiber layers to form a preform; and
(b) molding, wherein the act of molding comprises combining the preform with a molding material to form a molded preform.

2. The method of claim 1, wherein the act of preforming further comprises determining a desired orientation for the fiber when placing the fiber onto the substrate.

3. The method of claim 2, wherein the desired orientation for the fiber is determined by accounting for the stress that is expected to be experienced by component in use.

4. The method of claim 2, wherein the desired orientation for the fiber aligns the fiber parallel to the flux of force that is expected to be experienced by the fiber-reinforced component in use.

5. The method of claim 1, wherein the act of preforming further comprises determining a fiber type to be used when placing the fiber onto the substrate to form the fiber layer.

6. The method of claim 5, wherein the act of preforming further comprises combining a first fiber layer having a first fiber type with a second fiber layer having a second fiber type, wherein the first and second fiber types are different.

7. The method of claim 5, wherein the act of preforming further comprises placing fiber of multiple fiber types onto the substrate to form the fiber layer.

8. The method of claim 1, wherein the act of preforming further comprises draping the fiber layers over a core.

9. The method of claim 8, wherein the core directs the fibers of the fiber layers so the fibers are placed in a border area of the resulting fiber-reinforced component.

10. The method of claim 1, wherein the act of preforming further comprises adding an insert to the preform.

11. The method of claim 10, wherein the insert is configured to strengthen the resulting fiber-reinforced component.

12. The method of claim 10, wherein the insert is added at an interface area where multiple fiber-reinforced components are connectable.

13. The method of claim 1, wherein the substrate is removed before molding.

14. The method of claim 1, wherein the act of molding further comprises placing the preform in a mold cavity and injecting the mold cavity with the molding material.

15. The method of claim 14, wherein the act of molding further comprises curing the injected molding material under thermal energy, pressure, and time.

16. The method of claim 1, wherein the act of finishing further comprises connecting a first fiber-reinforced component with a second fiber-reinforced component to form an assembly of fiber-reinforced components.

17. The method of claim 1, further comprising finishing, wherein the act of finishing comprises smoothing the molded preform to form the fiber-reinforced component.

18. A head fixation device for use to stabilize a head of a patient in a medical procedure, the head fixation device comprising a frame, wherein at least a portion of the frame comprises a fiber-reinforced composite comprising fiber layers combined with a molding material, wherein the fiber of the fiber layers is configured to be oriented parallel to a flux of force experienced by the fiber-reinforced component in use.

19. The head fixation device of claim 18, wherein the frame comprises:
(a) a first arm;
(b) a second arm selectively and adjustably connectable with the first arm; and
(c) a pair of stabilizing assemblies, wherein one of the pair of stabilizing assemblies is selectively and adjustably connectable with the first arm, and wherein the other of the pair of stabilizing assemblies is selectively and adjustably connectable with the second arm.

20. The head fixation device of claim 18, wherein the fiber layers surround a core.
